(19) United States
(12) Patent Application Publication
(10) Pub. No.: US 2008/0138308 A1
(43) Pub. Date: Jun. 12, 2008

(54) Anhydrous Hair Conditioning Composition

(76) Inventors: Maria Teresa Belmar, Sharnbrook (GB); Shiping Zhu, Sharnbrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/662,232

(22) PCT Filed: Sep. 6, 2005
(86) PCT No.: PCT/EP05/09573

§ 371 Date: Oct. 3, 2007

(30) Foreign Application Priority Data
Sep. 9, 2004 (EP) 04225475.8

(51) Int. Cl. A61K 8/33; A61Q 5/12

(52) U.S. Cl. 424/70.19

(57) ABSTRACT

A base composition comprising non-ionic surfactant and cationic surfactant is provided. The base composition is prepared by heating the surfactant mixture in the substantial absence of other ingredients. The base composition is a simple composition which is suitable for use in preparing a hair conditioner composition by mixing with water

### FIELD OF THE INVENTION

The invention relates to a process for preparing a concentrate composition suitable for use in preparing a hair conditioner composition and hair conditioning customisation products.

### BACKGROUND TO THE INVENTION

Hair conditioning products provide several benefits to the user. Among these benefits are softness, control of static hair, softening of the hair. The efficacy of these products is influenced by various factors such as frequency of use, type of hair of the user, amount that is used and the specific composition of the conditioner.

Conventional hair conditioners are prepared by a process wherein a quaternary ammonium compound is mixed with compositions such as emollients, surfactants, oils, esters and the like and heated to a temperature of over 80° C. in the presence of water. Alternatively they are prepared in a process wherein ingredients are heated separately to a temperature over 80° C. and then mixed with water.

This method of preparation offers little flexibility to adapt the product formulation to the individual hair type of a client. Furthermore it is already disclosed in WO-A-03/037280 that the increase of the level of conditioning agents such as cationic surfactants may lead to problems in the manufacturing process and to instable products that suffer reduced spreadability.

To overcome this problem of the prior art compositions, WO-A-03/037280 provides an anhydrous cosmetic composition comprising a) a quaternary ammonium compound having at least one group selected from the group consisting of an ester group, an amido group and mixtures thereof and; b) an anhydrous carrier. The document further teaches that the presence of a multitude of other ingredients such as inorganic heat generating agents, polyoxyalkylene derivatives, reaction control agents, heat reserving material and hair conditioning compositions such as high melting point fatty compounds, additional cationic surfactants, cationic polymers, polypropylene glycols, oily conditioning agents is highly preferred. As a result the compositions according to the examples of this document contain a large number of ingredients and can be called complex mixtures of ingredients.

Furthermore DE19732015 discloses self-emulsifying compositions. These compositions are prepared by heating a mixture of a quaternary ammonium salt and an oil body and an emulsifier to a temperature higher than their melting point, and cooling them. In use they are mixed with water and optionally other ingredients.

Based on the above there is a desire for a simple, non-complex composition which is self-emulsifying and can be used as a suitable base composition for preparing a hair conditioner composition by the addition of water.

### SUMMARY OF THE INVENTION

We have surprisingly found that by preparing a mixture consisting essentially of a non-ionic surfactant and a cationic surfactant, heating this to a temperature higher than the melting point and cooling, a base composition is obtained which is suitable use in preparing a hair conditioner composition.

Therefore the invention relates to a process for preparing a base composition suitable for use in preparing a hair conditioner composition which process comprises the steps of
a) providing a mixture comprising at least one non-ionic surfactant and at least one cationic surfactant wherein the total amount of surfactant in the mixture is from 90 to 100 wt %,
b) bringing this mixture to a temperature above the melting temperature of the mixture,
c) cooling to a temperature below 30° C.

The invention further relates to a base composition obtainable by this process.

### DETAILED DESCRIPTION

In the context of the invention hair conditioning compositions are the same as hair care compositions. In this context hair conditioning compositions are compositions comprising ingredients such as cationic surfactant, non-ionic surfactant, silicon oils, mineral oils, pearlescent agent, perfumes, preservatives and the like.

The invention provides a process for preparing a base composition suitable for use in preparing a hair conditioner composition. The base composition can be supplemented with optional ingredients that are known to be included in conditioners. In use the base composition can be mixed with water and applied to hair or alternatively can be applied to wet or dry hair to mix with water on the spot.

In use the base composition can be mixed with water or with any other water containing liquid such as tea, fruit extracts, vegetable extracts or the like.

The composition is a simple mixture of few ingredients, which offers a cheap, easily prepared base composition for preparing a wide variety of hair conditioners. The variety can be introduced by selecting the amount of water that is added and by adding further ingredients to the base composition. Alternatively the base composition is used as such after mixing with water.

In a first step a mixture is provided comprising at least one non-ionic surfactant and at least one cationic surfactant.

The mixture comprises a cationic surfactant. The cationic surfactant is preferably a quaternary ammonium compound. Examples of suitable quaternary ammonium compounds are behenyltrimethylammonium chloride, dialkyldimethyl ammonium chlorides, cetyl trimethyl ammonium bromide or chloride and mixtures thereof. Alternative suitable quaternary ammonium compounds are disclosed in WO-A-03/037280.

The amount of cationic surfactant is preferably from 5 to 90 wt %, more preferably from 5 to 45 wt % on total weight of the mixture in step (a).

The mixture also comprises a non-ionic surfactant. The non-ionic surfactant is preferably selected from the group comprising polyoxyethylene glyceryl esters such as PEG 100 and PEG 20, fatty alcohols, fatty acids with a degree of neutralization below 5% or a combination thereof.

The preferred non-ionic surfactant is a fatty alcohol. Even more preferred the fatty alcohol is selected from the group comprising cetyl alcohol, stearyl alcohol, behenyl alcohol and mixtures thereof. The well known mixture of cetyl alcohol and stearyl alcohol; cetearyl alcohol, is highly preferred.

The amount of non-ionic surfactant in the mixture is preferably from 1 to 90 wt %, more preferred 5 to 70 wt %, even more preferably from 30 to 50 wt %.

The total amount of non-ionic surfactant plus cationic surfactant in the mixture is from 90 to 100 wt %, preferably 95 to 100 wt %, more preferably 98 to 100 wt % of the total mixture in step (a).

This mixture is subjected to heating. This heating step is essential to the invention because the mere admixing of these ingredients would not lead to a base composition that easily forms a homogeneous lamellar gel structure in water to form a hair conditioner. Therefore in step (b) the mixture is brought to a temperature above the melting point of the surfactant mixture. Generally the temperature in step (b) will be above 70° C., preferably from 70 to 160° C., more preferred from 75 to 100° C.

In step (c) the mixture is cooled to a temperature below 30° C., preferably below 20° C. The mixture then generally is in the form of a liquid, paste or powder. Optionally the product is brought into another physical state such as flakes, powder or pellets that are suitable for use. The powder form is highly preferred for ease of homogeneous dispersion in water.

Optionally the mixture in step (a) additionally comprises a compound selected from the group consisting of silicon oils, mineral oils, vegetable oils or a combination thereof. These compounds are known to be suitable for inclusion in a hair conditioner composition and are best added at the start of the process, before heating.

To further customize the final hair conditioning product, the process optionally comprises a step (d) wherein further ingredients are added to the composition of step (c). The further ingredients are preferably selected from the group comprising perfumes, preservatives, hair conditioning agents, colourants or a combination thereof. These optional components may suitably be added to the base composition but may also be added at a later stage after the base composition has been mixed with water.

In use the base composition is preferably mixed with water before use. The ratio of base composition to water will depend on the specific hair type of the end user. This base composition offers a wide variety in terms if final surfactant and hair conditioning ingredient. Preferably the dilution with water is such that the final concentration of non-ionic surfactant is from 1 to 10 wt % on total weight of the hair conditioning composition and the final concentration of cationic surfactant is from 1 to 10 wt % on total weight of the hair conditioning composition.

The base composition may be used by an individual consumer or alternatively could be used for small scale production facilities to make the production process easy and efficient.

The invention further relates to a base composition obtainable by the process according to the invention. This specific process of heating the non-ionic surfactant and cationic surfactant in a mixture which contains only up to 10 wt %, preferably only up to 5 wt % other ingredients leads to the formation of a base composition which has a structure such that dilution with water results in a hair conditioner with desired Theological properties.

Preferably the base composition according to the invention comprises less than 5 wt % water.

The invention is now illustrated by the following non limiting examples.

### EXAMPLES

### Example 1

TABLE 1
composition example 1

| Ingredient | Weight percentage % |
|---|---|
| Cetyltrimethylammonium | 65 |
| bromide | |
| Cetyl alcohol | 35 |

### Example 2

TABLE 2
composition example 2

| Ingredient | Weight percentage % |
|---|---|
| Cetyltrimethylammonium | 60 |
| bromide | |
| Stearic acid | 10 |
| Cetyl alcohol | 30 |

### Preparation Method:

Mixing the ingredients together and heating to about 100° C. to melt the surfactants (an isotropic mix is obtained). The melt is then cooled down to form a solid. A powder is made by grinding the solid.

To make a conditioner 5 wt % of the powder is mixed with 95 wt % water at temperatures between 10-90° C.

To make a customized product silicon oils, mineral oils, perfume and pearlescent agent etc. can be mixed with the powder and water together or added to a pre-made conditioner base at temperatures between 10-90° C.

1. Process for preparing a base composition suitable for use in preparing a hair conditioner composition which process comprises the steps of
a) providing a mixture comprising at least one non-ionic surfactant and at least one cationic surfactant wherein the total amount of surfactant in the mixture is from 90 to 100 wt %,
b) bringing this mixture to a temperature above the melting temperature of the mixture,
c) cooling to a temperature below 30° C.

2. Process according to claim 1 wherein the non-ionic surfactant is selected from the group comprising polyethylene glycol esters and ethers, fatty acids with a degree of neutralization below 5%, fatty alcohols or a mixture thereof.

3. Process according to claim 2 wherein the non-ionic surfactant is cetearyl alcohol.

4. Process according to claim 1 wherein the cationic surfactant is a quaternary ammonium compound.

5. Process according to claim 1 wherein the mixture in step (a) additionally comprises a compound selected from the group consisting of silicon oils, mineral oils, vegetable oils or a combination thereof.

6. Process according to claim 1 which additionally comprises a step (d) wherein further ingredients are added to the composition of step (c) and wherein the further ingredients are selected from the group comprising perfumes, preservatives, hair conditioning agents, colourants or a combination thereof.

7. Base composition obtainable by the process according to claim 1.

8. Base composition according to claim 7 which comprises less than 5 wt % water.
